(19) United States
(12) Patent Application Publication
(10) Pub. No.: US 2023/0038706 A1
(43) Pub. Date: Feb. 9, 2023

(54) PROCESS FOR PREPARING ALKYLENE GLYCOL MIXTURE FROM A CARBOHYDRATE SOURCE WITH INCREASED SELECTIVITY FOR GLYCEROL

(71) Applicant: Avantium Knowledge Centre B.V., Amsterdam (NL)

(72) Inventors: Davide Ansovini, Amsterdam (NL); Paula Claassens-Dekker, Amsterdam (NL); Jagdeep Singh, Amsterdam (NL)

(21) Appl. No.: 17/790,647

(22) PCT Filed: Feb. 8, 2021
(86) PCT No.: PCT/EP2021/052952

§ 371 Date: Jul. 1, 2022

(30) Foreign Application Priority Data
Feb. 17, 2020 (EP) 20157699.8

(51) Int. Cl. C07C 29/156; C07C 29/153

(52) U.S. Cl. CPC C07C 29/156; C07C 29/153

(57) ABSTRACT

The invention relates to a process for preparing a mixture of alkylene glycols (e.g. ethylene glycol and/or propylene glycol) from a carbohydrate source by catalytic conversion with hydrogen. More specifically, the catalytic hydrogenolysis process of the invention has an increased selectivity for glycerol, next to producing considerable amounts of ethyelene glycol and propylene glycol. This is achieved by ensuring the carbohydrate feed is rich in sucrose.

### INTRODUCTION

The present invention relates to a process for preparing a mixture of alkylene glycols (e.g. ethylene glycol and/or propylene glycol) from a carbohydrate source by catalytic conversion with hydrogen. More specifically, the catalytic hydrogenolysis process of the invention has an increased selectivity for glycerol (i.e. glycerol as part of the product mixture in increased amounts).

### BACKGROUND OF THE INVENTION

Alkylene glycols such as ethylene glycol and propylene glycol are valuable products or intermediates in chemical industry, as such compounds are used in various chemical processes. Traditionally, alkylene glycols are produced from fossile sources. More recently, there is ongoing research to produce alkylene glycols from renewable sources.

In this connection, CN 102643165 describes a process for producing ethylene glycol and propylene glycol from soluble sugars or starch. Similarly, US 7960594 discloses a process in which ethylene glycol is produced from cellulose. In WO 2016/114661 it is stated that ethylene glycol may be obtained from a carbohydrate source by catalytic reaction with hydrogen, which carbohydrate source may be obtained from a variety of sources, such as polysaccharides, oligosaccharides, disaccharides and monosaccharides (which all may be obtained from renewable sources). Suitable examples are stated to be cellulose, hemicellulose, starch, sugars such as sucrose, mannose, arabinose, glucose and mixtures thereof. Only glucose is exemplified as a starting material.

Most references such as US2011/0312488 and WO2017/097839 disclosing processes to obtain alkylene glycols like ethylene glycol or propylene glycol from (renewable) carbohydrates refer in a general sense to whole ranges of carbohydrates as potential starting material, but exemplify often only glucose, starch or cellulose as carbohydrate source.

R Ooms et al, RCS, Green Chemistry, Royal Society of Chemistry, vol 16 Jan. 1, 2014 pages 695-707 discloses batch and fed batch processes for the conversion of sugars to ethylene glycol using nickel tungsten carbide as catalyst.

WO2019/175362 discloses a continuous or semi-continuous process for the preparation of ethylene glycol. The catalyst system comprises a homogeneous catalyst containing tungsten and a heterogeneous catalyst containing one or more transition metals from group 8, 9 or 10 on a carrier. The substrate is glucose.

The processes in the above references and others generally produce ethylene glycol in a selectivity up to 50 to 60%, based on the carbohydrate feed, when glucose is used as a feed, next to a whole range of other components in varying amounts. Next to ethylene glycol, smaller amounts of propylene glycol are obtained in such reactions. Apart from these two desired products, lower alkanols, butanediol (both 1,2-and 1,4-), and polyols like glycerol, erythritol, and sorbitol are formed as side products in the product mix.

These other products are usually either obtained in amounts that are not commercially attractive, or are difficult to purify from the reaction mixture. However, there is a desire to obtain valuable components from the mixture produced next to ethylene glycol and propylene glycol, as that broadens commercial opportunities and can make a commercial operation commercially more robust. To achieve this, it is desired that glycerol is obtained by such hydrogenolysis process of renewable carbohydrates in commercially attractive amounts. Glycerol is generally present in such product mix, but the selectivity for such is usually too limited to warrant isolation of it. Thus, selectivity for glycerol in such hydrogenolysis should preferably be increased, when e.g. compared to the standard process using glucose as a starting material. Regarding the starting material, such should preferably be an abundantly available material of consistent quality and composition.

Hence, there is a need for a process for preparing a mixture of alkylene glycols from carbohydrates by reaction with hydrogen, in which reaction next to yielding economically attractive amounts of valuable smaller alkane glycols like ethylene glycol and propylene glycol, glycerol is obtained in considerable amount. Hence, there is a need for a process for preparing alkylene glycols from carbohydrates by reaction with an increased selectivity for glycerol, when compared to using glucose as a starting material. Next to glycerol, such process preferably should also yield considerable amounts of ethylene glycol and propylene glycol, as such components can be separated off from the product mix easily. Preferably, the process should be able to be carried out in a continuous way.

### SUMMARY OF THE INVENTION

It has now been found that the above objective can be met, at least in part, by a process for producing a mixture of 2 to 40% by weight of glycols dissolved in water with a ratio of between 1: 0.2 to 1: 8 for the selectivity for glycerol : the combined selectivity for ethylene glycol and propylene glycol, of the reaction products produced by said process, which process comprises feeding to a pressurized, continuously stirred tank reactor hydrogen and an aqueous feed solution comprising water and a carbohydrate, wherein the reactor contains a catalyst system which comprises a homogeneous catalyst comprising a tungsten compound and a heterogeneous catalyst comprising a hydrogenolysis metal selected from the groups 8, 9 or 10 of the Periodic Table of the Elements, characterized in that the carbohydrate in the aqueous feed comprises at least 80% of sucrose, by weight based on the total amount of carbohydrate in the aqueous feed.

In other words, it was found that the amount of a desired component like glycerol, which is a valuable byproduct from hydrogenolysis of carbohydrates using hydrogen and a catalyst system comprising a homogeneous catalyst and a heterogeneous catalyst can be increased if one ensures that the feed of carbohydrates comprise a substantial amount of sucrose (e.g. at least 80% on the weight of carbohydrates in the feed). Or put differently, the selectivity for glycerol can be increased if the carbohydrate feed comprises a substantial amount of sucrose, whilst still producing attractive amounts of smaller alkylene glycols like ethylene glycol and propylene glycol. The present invention is such that sucrose can directly (i.e. without the need for hydrolysis into the monosaccharides glucose and fructose) be fed to the reactor (in solution).

### DETAILED DESCRIPTION OF THE INVENTION

Depending on the amount of sucrose in the feed and detailed reaction conditions commercially attractive amounts of glycerol can be produced, whilst still maintaining a good yield on smaller alkylene glycols like ethylene glycol and propylene glycol. Hence, in the present invention it is preferred that the selectivity for glycerol : the combined selectivity for ethylene glycol and propylene glycol of the reaction products produced by said process is between 1:1 and 1:6.

To this end, and also for reasons of easier handling of the feed, it is preferred that the carbohydrate in the aqueous feed comprises at least 90% of sucrose, preferably at least 95% by weight of sucrose, by weight based on the total amount of carbohydrate in the aqueous feed. Most preferably, the feed is only sucrose, but in industrial sucrose minor amounts (e.g. 1-5% by weight) of other carbohydrates can still be present, which are not detrimental to the outcome. The reaction is preferably a continuous process, and the aqueous feed solution comprising water and a carbohydrate in the present process preferably comprises between 5 and 35% by weight of carbohydrate, preferably between 10 and 30% by weight of carbohydrate (by weight on the total feed).

As stated, the carbohydrate source containing sucrose is converted into a product mix comprising ethylene glycol and propylene glycol with hydrogen and a catalyst system which comprises a homogeneous catalyst comprising a tungsten compound and a heterogeneous catalyst comprising a hydrogenolysis metal selected from the groups 8, 9 or 10 of the Periodic Table of the Elements. Concerning the latter, it is preferred that the hydrogenolysis metal from groups 8, 9 or 10 of the Periodic Table of the Elements in this connection is selected from the group consisting of Cu, Fe, Ni, Co, Pd, Pt, Ru, Rh, Ir, Os and combinations thereof. Ruthenium is the preferred metal selected from the groups 8, 9 or 10 of the Periodic Table of the Elements in the present invention. It is preferred in the present invention that the amount of the hydrogenolysis metal selected from the groups 8, 9 or 10 of the Periodic Table of the Elements which present in the reactor is preferably present in an amount of between 0.05 and 20 g hydrogenolysis metal / L of reactor volume, more preferably between 0.1 and 12 g g hydrogenolysis metal / L of reactor volume, and most preferably between 0.5 and 8 g hydrogenolysis metal / L of reactor volume.

The hydrogenolysis metal catalyst referred to above can be present as such, but it is preferred that such is present in the form of a catalyst supported on a carrier. Preferred carriers in this case are carriers selected from the group supports, consisting of activated carbon, silica, alumina, silica-alumina, zirconia, titania, niobia, iron oxide, tin oxide, zinc oxide, silica-zirconia, zeolites, aluminosilicates, titanosilicates, magnesia, silicon carbide, clays and combinations thereof. Activated carbon is a preferred carrier in the present invention, in particular with the hydrogenolysis catalyst being ruthenium.

Next to the metal selected from groups 8, 9 or 10 of the Periodic Table of the Elements (the heterogeneous catalyst part) the catalyst system comprises a homogeneous catalyst part, which is herein a tungsten compound. In the process according to the present invention, it is preferred that the homogeneous catalyst comprising a tungsten compound is selected from the group consisting of tungstic acid (H₂WO₄), ammonium tungstate, ammonium metatungstate, ammonium paratungstate, tungstate compounds comprising at least one Group 1 or 2 element, metatungstate compounds comprising at least one Group 1 or 2 element, paratungstate compounds comprising at least one Group 1 or 2 element, tungsten oxide (WO₃), heteropoly compounds of tungsten, and combinations thereof. A most preferred homogeneous catalyst in the present reaction comprises tungstic acid and/or a tungstate, e.g. ammonium tungstate, sodium tungstate or potassium tungstate. The homogeneous catalyst comprising a tungsten compound in the present invention is preferably dissolved or dispersed in water and/or an alkylene glycol, the latter preferably being ethylene glycol.

The presently claimed process is preferably carried out as a continuous process. To this end, to the reactor are continuously or periodically added: a stream comprising a carbohydrate feed, and the same for pressurized hydrogen gas. Preferably also the homogeneous catalyst comprising a tungsten compound is continuously or periodically added to the reactor. The amount of catalyst in the feed to the reactor is preferably such that the concentration of the homogeneous catalyst comprising a tungsten compound present in the reactor is between 0.05 and 5 wt.%, preferably between 0.1 and 2 wt.% calculated as tungsten metal.

In the present invention the amount of the homogeneous catalyst comprising a tungsten compound and a heterogeneous catalyst comprising a hydrogenolysis metal selected from the groups 8, 9 or 10 of the Periodic Table of the Elements are present in the reactor in amounts such that the weight ratio of weight of tungsten to the total weight of hydrogenolysis metal, all calculated on metal basis, is between 1: 3000 to 50 : 1 (tungsten metal : transition metal wt : wt).

The process of the present invention is carried out at elevated pressure (i.e. higher than atmospheric). Preferably, the total pressure in the reactor is between 2.0 and 16 MPa, preferably between 4 and 12 MPa, most preferably between 5 and 10 MPa. As hydrogen is key to the present reaction, pressurization is preferably carried out with hydrogen. Furthermore, it is preferred in the present invention that the aqueous feed solution comprising water and a carbohydrate comprises between 40% and 85% of water, between 5 and 35% by weight of carbohydrate, and between 5 to 40% of an alkylene glycol co-solvent (preferably ethylene glycol), all by weight on the total aqueous feed solution.

The reaction is preferably carried out such that the temperature in the reactor is between 150 and 270° C., preferably between 180 and 250° C. The rate of addition of aqueous feed solution comprising water and a carbohydrate into the CSTR is such that WHSV is preferably between 0.01 and 100 hr⁻¹, preferably between 0.05 and 10 hr⁻¹, more preferably between 0.5 and 2 hr⁻¹.

The invention further relates to a process for producing glycols dissolved in water, which process comprises feeding to a pressurized, continuously stirred tank reactor hydrogen an aqueous feed solution comprising water and a carbohydrate, wherein the reactor comprises a catalyst system which comprises a homogeneous catalyst comprising a tungstic acid and a heterogeneous catalyst comprising ruthenium, characterized in that the carbohydrate in the aqueous feed comprises at least 80% of sucrose, by weight based on the total amount of carbohydrate in the aqueous feed, as it was found that the above objective can be met, at least in part, by this process. In this process, it is preferred the heterogeneous catalyst comprises ruthenium supported on a carrier. Preferred carriers in this case are carriers selected from the group supports, consisting of activated carbon, silica, alumina, silica-alumina, zirconia, titania, niobia, iron oxide, tin oxide, zinc oxide, silica-zirconia, zeolites, aluminosilicates, titanosilicates, magnesia, silicon carbide, clays and combinations thereof. Activated carbon is a preferred carrier in the present invention.

The presently claimed process is preferably carried out as a continuous process. To this end, to the reactor are continuously or periodically added: a stream comprising a carbohydrate feed, and the same for pressurized hydrogen gas. Preferably also the homogeneous catalyst comprising a tungsten compound is continuously or periodically added to the reactor. The amount of catalyst in the feed to the reactor is preferably such that the concentration of the homogeneous catalyst comprising tungstic acid present in the reactor is between 0.05 and 5 wt.%, preferably between 0.1 and 2 wt.% calculated as tungsten metal.

It is preferred in the present invention that the amount of ruthenium which present in the reactor is preferably present in an amount of between 0.05 and 20 g ruthenium / L of reactor volume, more preferably between 0.1 and 12 g ruthenium / L of reactor volume, and most preferably between 0.5 and 8 g ruthenium / L of reactor volume. The amount of the homogeneous catalyst comprising tungstic acid and a heterogeneous catalyst comprising ruthenium are present in the reactor in amounts such that the weight ratio of weight of tungsten to the total weight of hydrogenolysis metal, all calculated on metal basis, is between 1: 3000 to 50 : 1 (tungsten metal : transition metal wt : wt).

In the above process, the homogeneous catalyst comprising a tungstic acid is preferably dissolved or dispersed in water and/or an alkylene glycol, the latter preferably being ethylene glycol.

In this process, the carbohydrate in the aqueous feed preferably comprises at least 90% of sucrose, more preferably at least 95% by weight of sucrose, by weight based on the total amount of carbohydrate in the aqueous feed. Furthermore, it is preferred that in the now claimed process the aqueous feed solution comprising water and a carbohydrate preferably comprises between 5 and 35% by weight of carbohydrate, preferably between 10 and 30% by weight of carbohydrate.

The process of the present invention is carried out at elevated pressure (i.e. higher than atmospheric). Preferably, the total pressure in the reactor is between 2.0 and 16 MPa, preferably between 4 and 12 MPa, most preferably between 5 and 10 MPa. As hydrogen is key to the present reaction, pressurization is preferably carried out with hydrogen. Furthermore, it is preferred in the present invention that the aqueous feed solution comprising water and a carbohydrate comprises between 40% and 85% of water, between 5 and 35% by weight of carbohydrate, and between 5 to 40% of an alkylene glycol co-solvent (preferably ethylene glycol), all by weight on the total aqueous feed solution.

The reaction is preferably carried out such that the temperature in the reactor is between 150 and 270° C., preferably between 180 and 250° C. The rate of addition of aqueous feed solution comprising water and a carbohydrate into the CSTR is such that WHSV is preferably between 0.01 and 100 hr⁻¹, preferably between 0.05 and 10 hr⁻¹, more preferably between 0.5 and 2 hr⁻¹.

### EXAMPLES

### Example 1: Sucrose Purity > 99% As Feed Carbohydrate in Hydrogenolysis

### Comparative 1: Glucose Purity > 99% As Feed Carbohydrate in Hydrogenolysis

### Process Description

Hydrogenolysis experiments were carried out in a continuously stirred tank reactor. The amount of liquid in the reactor was about 170 ml. Trials were done with two different residence times:
about 24 minutes residence time (example 1a and comparative 1a) and
about 34 minutes residence time (example 1b and comparative 1b).

The reactor contained as heterogeneous catalyst ruthenium on activated carbon. The amount of ruthenium on activated carbon was about 5 wt% Ru on AC. The total weight of heterogeneous catalyst on carrier was about 7 g Ru + AC for example 1a and comparative 1a, and about 4.3 g for Ru + AC for example 1b and comparative 1b. The reactor was filled with Ru/AC and water before the reactor was heated and pressurized. All of the heterogeneous catalyst remained in the reactor during the reaction.

The carbohydrate feed was prepared by dissolving the sucrose (examples 1a and 1b) and glucose (comparatives 1a and 1b) in a mixture of water and ethylene glycol at a concentration of about 20 wt% on the final feed composition which further contained about 60 wt% water and 20 wt% ethylene glycol.

The homogeneous catalyst solution was prepared by dissolving sodium hydroxide and H₂WO₄ in ethylene glycol, at a molar ratio of 0.7 : 1, to arrive at a concentration H₂WO₄ of 0.44 wt %.

The carbohydrate feed solution and homogeneous catalyst solution were mixed prior to use.

The reactor was heated to 220° C. and pressurised with hydrogen gas to 65 bar. Hydrogen gas was entered into the reactor at a flow of 2000 ml/minute.

At the start of the reaction (t = 0 minutes) the mixture of carbohydrate feed and homogeneous catalyst solution was pumped into the reactor at a steady flow to obtain the residence times indicated (flow rates added to the reactor at 5 or 7 ml per minute.

Reactions were carried out for about 300 minutes, and from the outlet stream samples were taken at 8 -10 times in the interval from 0 to 300 minutes.

### Results

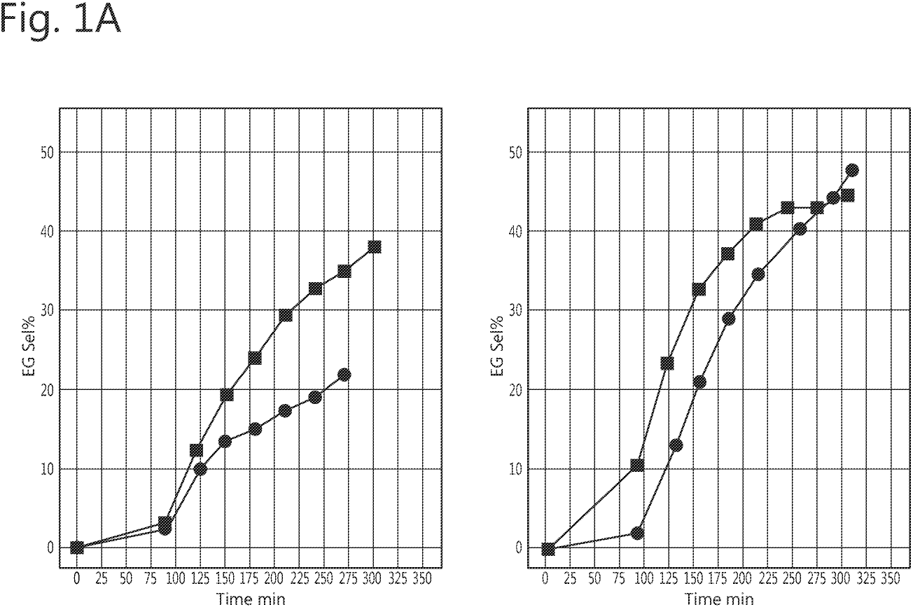

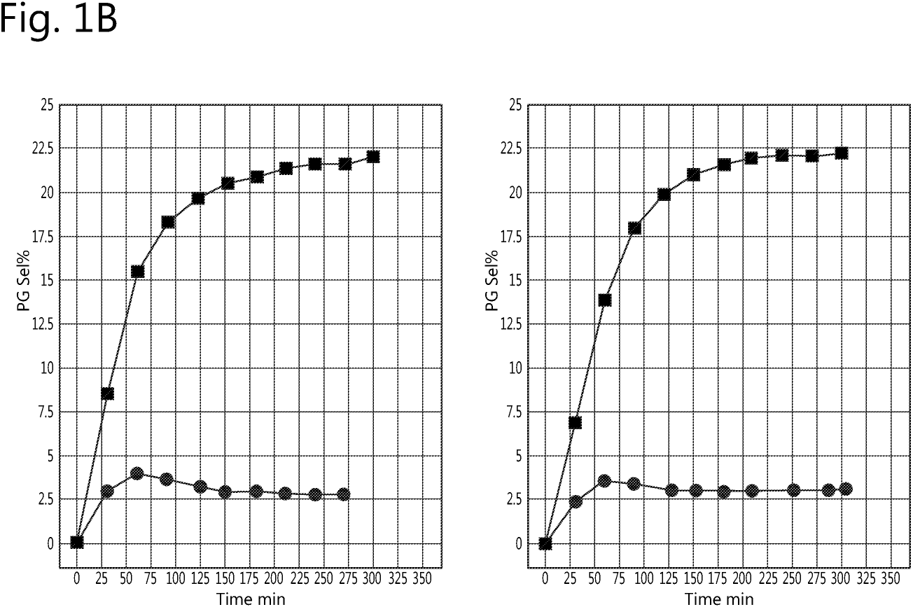

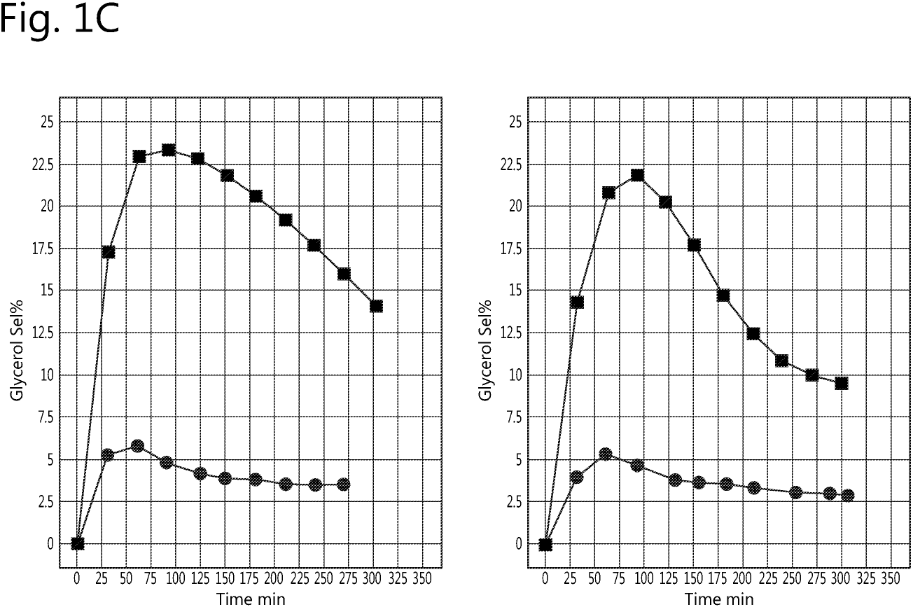

The samples obtained were analysed on concentration of polyol (ethylene glycol, propylene glycol, and glycerol) using HPLC and from this reaction selectivities were calculated. The results are set out in FIGS. 1A to 1C.

FIG. 1A: selectivity of ethylene glycol obtained in the product stream, for sucrose as feed (squares) and glucose as feed (circles) for a residence time of about 24 minutes (left hand) and for a residence time of about 34 minutes (right hand).

FIG. 1B: selectivity of propylene glycol obtained in the product stream, for sucrose as feed (squares) and glucose as feed (circles) for a residence time of about 24 minutes (left hand) and for a residence time of about 34 minutes (right hand).

FIG. 1C: selectivity of glycerol obtained in the product stream, for sucrose as feed (squares) and glucose as feed (circles) for a residence time of about 24 minutes (left hand) and for a residence time of about 34 minutes (right hand).

From the figures it can be concluded that when using sucrose as a feed a substantial higher selectivity is obtained for glycerol as opposed to the combined selectivity for ethylene glycol and propylene glycol, when compared to the selectivities for glucose as a feed.

1. A process for producing a mixture of 2 to 40% by weight of glycols dissolved in water with a ratio of between 1 : 0.2 to 1 : 8 for the selectivity for glycerol : the combined selectivity for ethylene glycol and propylene glycol, of the reaction products produced by said process, the process comprises:
feeding to a pressurized, continuously stirred tank reactor hydrogen and an aqueous feed solution comprising water and a carbohydrate, wherein the reactor contains a catalyst system which comprises a homogeneous catalyst comprising a tungsten compound and a heterogeneous catalyst comprising a hydrogenolysis metal selected from the groups 8, 9 or 10 of the Periodic Table of the Elements, wherein the carbohydrate in the aqueous feed comprises at least 80% of sucrose, by weight based on the total amount of carbohydrate in the aqueous feed.

2. The process according to claim 1, wherein the ratio for the selectivity for glycerol : the combined selectivity for ethylene glycol and propylene glycol of the reaction products produced by said process is between 1 : 1 and 1 : 6.

3. The process according to claim 1 , wherein the carbohydrate in the aqueous feed comprises at least 90% of sucrose, weight based on the total amount of carbohydrate in the aqueous feed.

4. The process according to claim 1 , wherein the aqueous feed solution comprising water and a carbohydrate comprises between 5 and 35% by weight of carbohydrate.

5. The process according to claim 1 , wherein the hydrogenolysis metal from groups 8, 9 or 10 of the Periodic Table of the Elements is selected from the group consisting of Cu, Fe, Ni, Co, Pd, Pt, Ru, Rh, Ir, Os and combinations thereof, .

6. The process according to claim 1 ,wherein the amount of the hydrogenolysis metal selected from the groups 8, 9 or 10 of the Periodic Table of the Elements present in the reactors is between 0.05 and 20 g hydrogenolysis metal / L of reactor volume.

7. The process according to claim 1 , wherein the hydrogenolysis metal from groups 8, 9 or 10 of the Periodic Table of the Elements is present in the form of a catalyst supported on a carrier, .

8. The process according to claim 1 ,wherein the homogeneous catalyst comprising a tungsten compound is selected from the group consisting of tungstic acid ,ammonium tungstate, ammonium metatungstate, ammonium paratungstate, tungstate compounds comprising at least one Group 1 or 2 element, metatungstate compounds comprising at least one Group 1 or 2 element, paratungstate compounds comprising at least one Group 1 or 2 element, tungsten oxide heteropoly compounds of tungsten, and combinations thereof.

9. The process according to claim 1 , wherein the homogeneous catalyst comprising a tungsten compound is dissolved or dispersed in water and/or an alkylene glycol.

10. The process according to claim 1 ,wherein the homogeneous catalyst comprising a tungsten compound is continuously or periodically added to the reactor.

11. The process according to claim 1 , wherein the amount of homogeneous catalyst present in the reactor is between 0.05 and 5 wt.%, calculated as tungsten metal.

12. The process according to claim 1 , wherein the homogeneous catalyst comprising a tungsten compound and a heterogeneous catalyst comprising a hydrogenolysis metal selected from the groups 8, 9 or 10 of the Periodic Table of the Elements are present in the reactor in amounts such that the weight ratio of weight of tungsten to the total weight of hydrogenolysis metal, all calculated on metal basis, is between 1: 3000 to 50 : 1 (tungsten metal : transition metal wt : wt).

13. The process for producing glycols dissolved in water, the process comprises: feeding to a pressurized, continuously stirred tank reactor hydrogen an aqueous feed solution comprising water and a carbohydrate, wherein the reactor comprises a catalyst system which comprises a homogeneous catalyst comprising a tungstic acid and a heterogeneous catalyst comprising ruthenium, wherein carbohydrate in the aqueous feed comprises at least 80% of sucrose, by weight based on the total amount of carbohydrate in the aqueous feed.

14. The process according to claim 13, wherein the heterogeneous catalyst comprises ruthenium supported on a carrier.

15. The process according to claim 13 , wherein the aqueous feed solution comprising water and a carbohydrate comprises between 40% and 85% of water, between 5 and 35% by weight of carbohydrate, and between 5 to 40% of an alkylene glycol co-solvent, all by weight on the total aqueous feed solution.
